(19) United States
(12) Patent Application Publication
(10) Pub. No.: US 2025/0255569 A1
(43) Pub. Date: Aug. 14, 2025

(54) SYSTEMS AND METHODS OF PROCESSING IMAGES OF EPICARDIAL AND PERICORONARY FAT

(71) Applicant: HeartFlow, Inc., Mountain View, CA (US)

(72) Inventors: Nan XIAO, San Jose, CA (US); Matthew SINCLAIR, London (GB); Sabrina LYNCH, Cupertino, CA (US); Michiel SCHAAP, Oegstgeest (NL); Souma SENGUPTA, Cupertino, CA (US); Timothy A. FONTE, San Francisco, CA (US); Adam UPDEPAC, Alamo, CA (US)

(21) Appl. No.: 19/046,742

(22) Filed: Feb. 6, 2025

Related U.S. Application Data
(60) Provisional application No. 63/551,321, filed on Feb. 8, 2024.

(51) Int. Cl. A61B 6/00; A61B 6/50; G06T 7/00; G06T 17/00; G16H 50/30

(52) U.S. Cl. CPC A61B 6/5217; A61B 6/504; A61B 6/507; G06T 7/0014; G06T 17/00; G16H 50/30; G06T 2207/10081; G06T 2207/20036; G06T 2207/30048; G06T 2207/30101

(57) ABSTRACT

A computer-implemented method for processing medical images may comprise: receiving image data for a patient; based on the received image data, determining: a patient-specific epicardial adipose tissue (EAT) metric or a patient-specific pericoronary adipose tissue (PCAT) metric, and at least one other patient-specific metric, and using the EAT metric or the PCAT metric, and the at least one other patient-specific metric, to determine a risk score for the patient or to classify a disease state of the patient.

### CROSS-REFERENCE TO RELATED APPLICATION

This application claims priority to U.S. Provisional Application No. 63/551,321, filed Feb. 8, 2024, which is incorporated herein by reference in its entirety.

### TECHNICAL FIELD

Embodiments disclosed herein may relate to systems and methods for image processing and, more particularly, systems and methods of processing images of epicardial and pericoronary fat.

### BACKGROUND

Various invasive techniques for obtaining cardiovascular information for a patient exist, and these invasive techniques have been used to investigate, diagnose, or otherwise characterize cardiovascular information for a patient. Although the information gathered using such invasive techniques may aid in making medical determinations pertaining to patient-related cardiovascular disease, these methods are limited and may present significant risks to the health of the patient being diagnosed.

To circumvent these shortcomings, less invasive methodologies (e.g., medical imaging techniques) may be used to produce images of subject anatomy and to make medical determinations and diagnoses. The images may be processed and used by downstream tasks. Such downstream tasks may include, for example, analysis, processing, or interpretation. However, a need exists for improved non-invasive techniques of analyzing and characterizing cardiovascular disease (CVD) and associated conditions in patients and subjects.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to certain aspects of the disclosure, systems and methods are disclosed for processing medical images of epicardial fat, e.g., epicardial adipose tissue (EAT), and pericoronary fat, e.g., pericoronary adipose tissue (PCAT).

In an example, a computer-implemented method for processing medical images may comprise: receiving image data for a patient; based on the received image data, determining: a patient-specific epicardial adipose tissue (EAT) metric or a patient-specific pericoronary adipose tissue (PCAT) metric; and at least one other patient-specific metric; and using the EAT metric or the PCAT metric, and the at least one other patient-specific metric, to determine a risk score for the patient or to classify a disease state of the patient.

Any of the methods, systems, or devices disclosed herein may include any of the following features. The at least one other patient-specific metric may include a vessel geometry, a vessel morphology, a plaque characteristic, or a hemodynamic measurement. The image data may be a first image data, the EAT metric may be a first EAT metric, the PCAT metric may be a first PCAT metric, and the at least one other patient-specific metric is at least a first other patient-specific metric, and, before determining the risk score or classifying the disease state of the patient, the computer-implemented method may further comprise: receiving second image data for the patient; based on the received second image data, determining: a second patient-specific EAT metric or a second patient-specific PCAT metric; and at least a second other patient-specific metric; and using the second EAT metric or the second PCAT metric, and the second other patient-specific metric to determine the risk score for the patient or to classify the disease state of the patient. The received image data may be used to generate a three-dimensional model of a vasculature of the patient. The computer-implemented method may further include generating a display image of the three-dimensional model, wherein the display image includes a color-coded indicator of the risk score or the disease state. The disease state may be Ischemia with Non-Obstructive Coronary Arteries (INOCA). The risk score may be predictive of a fractional flow reserve (FFR) value. Both the EAT metric and the PCAT metric may be used to determine the risk score for the patient or to classify the disease state of the patient.

In another example, a system for processing medical images of a patient may comprise: a data storage device storing instructions for medical image processing; and a processor configured to execute the instructions to perform operations comprising: receiving medical images of the patient; based on the received medical images, determining: a patient-specific epicardial adipose tissue (EAT) metric or a patient-specific pericoronary adipose tissue (PCAT) metric; and at least one other patient-specific metric; and using the EAT metric or the PCAT metric, and at least one other patient-specific metric, to determine a risk score for the patient or to classify a disease state of the patient.

In another example, a non-transitory computer-readable medium may store instructions that, when executed by one or more processors, cause the one or more processors to perform a computer-implemented method for medical image processing, and the computer-implemented method may comprise: receiving image data for a patient; based on the received image data, determining: a patient-specific epicardial adipose tissue (EAT) metric or a patient-specific pericoronary adipose tissue (PCAT) metric; and at least one other patient-specific metric; and using the EAT metric or the PCAT metric, and the at least one other patient-specific metric, to determine a risk score for the patient or to classify a disease state of the patient.

Additional objects and advantages of the techniques presented herein will be set forth in part in the description that follows, and in part will be apparent from the description, or may be learned by practice of the techniques presented herein. The objects and advantages of the techniques presented herein will be realized and attained by means of the elements and combinations particularly pointed out in the appended claims.

It is to be understood that both the foregoing general description and the following detailed description are exemplary and explanatory only and are not restrictive of the disclosure.

### BRIEF DESCRIPTION OF THE DRAWINGS

The accompanying drawings, which are incorporated in and constitute part of this specification, illustrate various exemplary embodiments and together with the description, serve to explain the principles of the disclosed embodiments.

FIG. 1 depicts a computer system or environment for performing the various techniques described herein.

FIG. 2A illustrates an example of a workflow that involves training a machine-learning model for processing medical images.

FIG. 2B illustrates an example of a medical image processing workflow.

FIG. 3A illustrates an example of a workflow that involves training a machine-learning model for processing medical images.

FIG. 3B illustrates an example of a medical image processing workflow.

FIG. 4 illustrates an example of a medical image processing workflow.

FIG. 5A illustrates an example of a workflow that involves training a machine-learning model for processing medical images.

FIG. 5B illustrates an example of a medical image processing workflow.

FIG. 6 illustrates an example of a medical image processing workflow.

FIG. 7 depicts an exemplary system that may execute techniques presented herein.

### DETAILED DESCRIPTION

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” means “and/or,” such that any listed item may be recited in combination or in the alternative. Relative terms, such as, “substantially” and “generally,” are used to indicate a possible variation of +10% of a stated or understood value.

As used herein, the terms “patient” and “subject” are used interchangeably and shall be understood to encompass any user of the method, system, or non-transitory computer-readable medium being described.

Medical imaging, and other methods of acquiring medical information, may involve receiving data through the use of various types of equipment. For example, medical imaging modalities may include contrast computed tomography (CT) (e.g., coronary computed tomography angiography, CCTA) or non-contrast CT. As further examples, spectral CT or dual layer CT may be used to improve the quantification of various metrics described herein. For example, using spectral CT or dual layer CT, (e.g. an attenuation curve) may improve quantification of PCAT, characterize material properties of the fat tissue more faithfully, or characterize changes on the macro-scale more relevant to disease (e.g. water in the fat or tissue).

During medical diagnostic events, data may be acquired using various acquisition protocols, with the protocol that is to be used being selected by a user based on the medical information that is desired. Acquired data, which may be in the form of medical images, may be processed and may be used to generate a three-dimensional model. The three-dimensional model may be displayed to the user in a way that facilitates user interpretation. For example, the model may include color-coded portions, shaded portions, annotations, or other indicators.

When receiving and processing medical information, it may be advantageous to utilize data that is indicative of a presence of a particular physiological state, such as inflammation. For example, imaging features derived from epicardial adipose tissue (EAT) and images derived from pericoronary adipose tissue (PCAT) may be utilized as biomarkers for inflammation (e.g., cardiovascular inflammation). Whole-heart EAT volume and PCAT attenuation may be linked to coronary disease severity, disease progression, and long term outcomes. Further, high coronary artery calcium (CAC) levels (e.g., ≥100 AU) and high EAT volume levels (e.g., ≥113 cm³) may be linked to lower survivability rates among patient populations. As used herein, “epicardial adipose tissue” (EAT) refers to adipose tissue between the myocardial surface and the visceral layer of the pericardium. EAT may not be separated by a fascia layer from cardiac muscle cells. Further, as used herein, “pericoronary adipose tissue” (PCAT) refers to the part of the EAT surrounding the coronary arteries and that is contiguous with parts of the adventitial layer of the vessel wall. PCAT may also be referred to as coronary perivascular adipose tissue (PVAT). Paracardial adipose tissue is attached to the outside of the parietal layer of the pericardium. Paracardial fat, combined with EAT, is referred to as intrathoracic fat. Pericardial fat may refer to intrathoracic fat or to a layer of fat outside the EAT between the visceral and parietal layers.

Under typical conditions, EAT may provide an energy source for the myocardium during increased metabolic demand, may produce anti-inflammatory and anti-atherogenic cardio-protective factors (e.g., adrenomedullin), and may regulate vascular tone. However, in the presence of risk factors, EAT may become hypertrophic, pro-inflammatory, pro-atherosclerotic, and may release pro-inflammatory substances directly into the coronary lumen. Further, vascular inflammation may be communicated to a patient's surrounding PCAT, thereby triggering local chronic inflammation. The inflammation may cause adipocytes to not uptake lipids as effectively, thereby reducing the adipocytes in terms of size and maturity. The reduction in size and maturity of the adipocytes may shift the balance between the lipid and aqueous phases of the PCAT. Expression of adipogenic genes, the average adipocyte size, and lipid accumulation in PCAT may all be driven by paracrine inflammatory signals from the vascular wall. Such biological effects of vascular inflammation on PVAT may be tracked by analyzing imaging sequences obtained during imaging events.

Thus, the utilization of imaging features that are derived from EAT and from PCAT may provide useful data for medical image analyses. Additionally, using CT imaging techniques, measures of EAT volume and PCAT attenuation may be used to complement other metrics, such as plaque measures and hemodynamics for risk stratification.

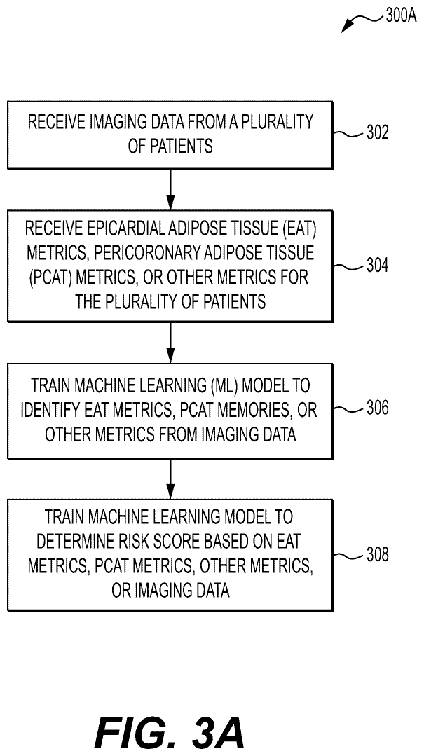

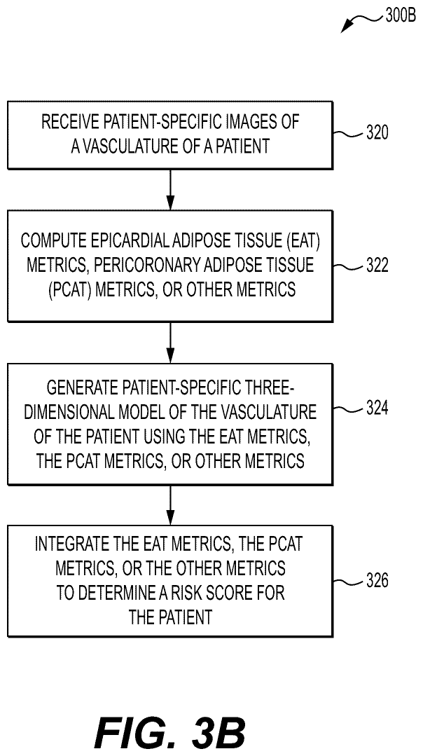

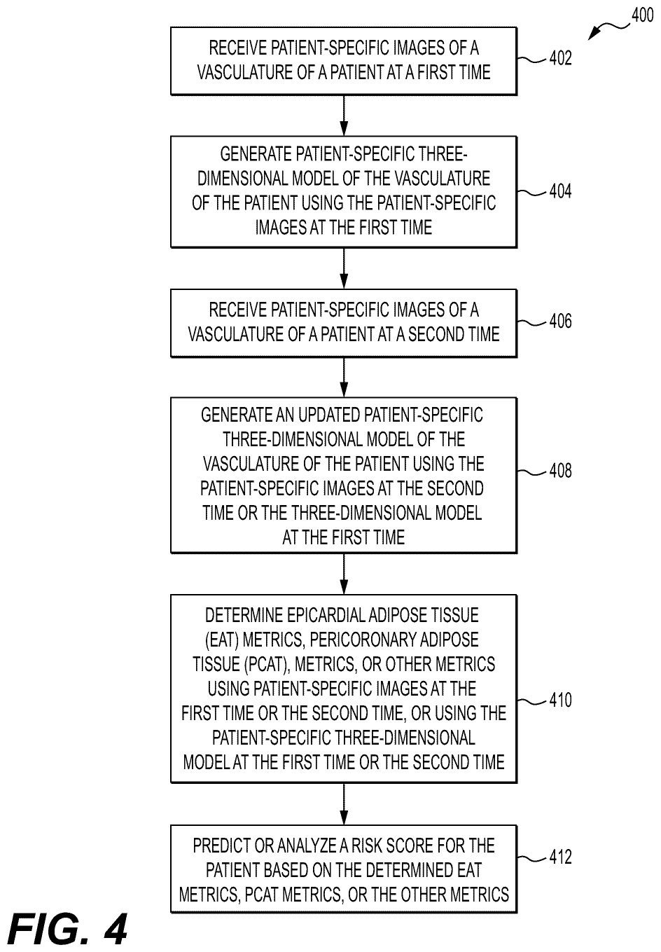

Accordingly, and as will be described in more detail herein in reference to, among others, FIGS. 3A-3B and 4, the systems and methods disclosed herein may be used for risk stratification or for the prediction of adverse outcomes. EAT volume and mean PCAT attenuation in proximal main vessels may have independent value in stratifying risk of adverse cardiovascular events. Lesion-based PCAT attenuation may be higher in acute coronary syndrome (ACS) than in coronary artery disease (CAD) and around soft plaque as opposed to calcified plaque. Integration of image-based, lesion-specific measurements of PCAT, plaque geometry and type, and hemodynamics, together with patient-level whole-heart EAT metrics and non-imaging-based risk factors may be used to determine which patients are at risk of events, and which lesions are more at risk. CT inflammation imaging (generating, e.g., EAT metrics and PCAT attenuation) may be combined with hemodynamics/computational physiology (e.g., FFRct, delta FFRct, myocardium-at-risk (MAR), plaque stresses), clinical risk factors (e.g., PREVENT or atherosclerotic cardiovascular disease (ASCVD) risk calculation), or CT plaque imaging (e.g., plaque volumes, burden, calcium score, adverse plaque characteristics (ACP)) to generate an overall risk score.

For example, contrast CT may be used to receive medical images of a patient, and EAT volume and PCAT attenuation may provide added value over existing image-based features and clinical risk factors. As another example, non-contrast CT may be used, and proximal main vessel PCAT, PCAT attenuation, or EAT metrics may be measured. In these examples, EAT and PCAT may provide added value over or in addition to existing techniques and metrics, such as calcium scoring. Total EAT volume extracted from non-contrast CT may have long-term prognostic value in addition to calcium scoring. From CCTA, right coronary artery (RCA) mean PCAT attenuation may be independently associated with outcomes after adjusting for clinical risk factors (relative to other imaging measures, such as calcium scoring and plaque).

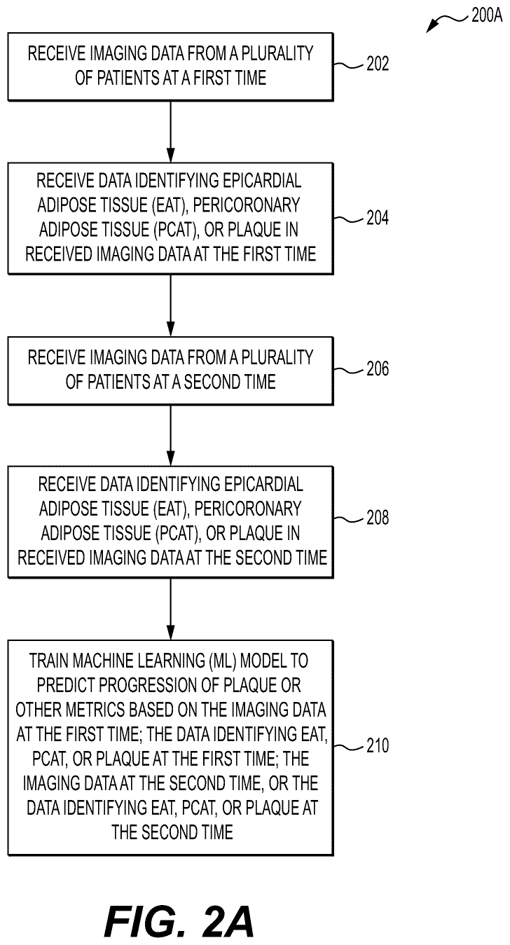

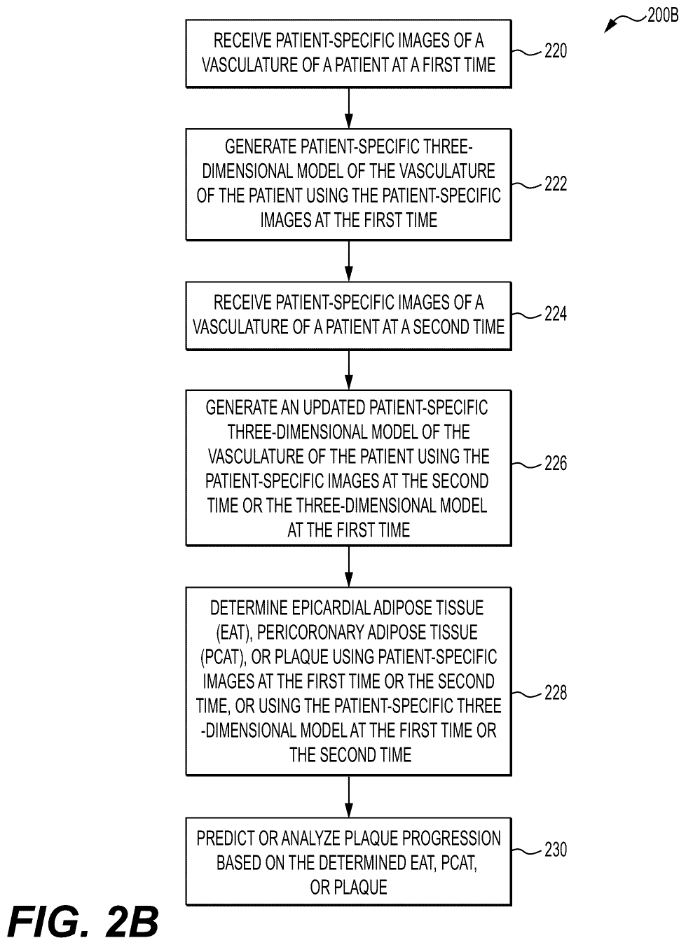

Further, and as will be described in more detail herein in reference to FIG. 2A and FIG. 2B, the systems and methods described herein may be utilized to predict plaque progression for a patient or a subject. Mean PCAT attenuation measured around the right coronary artery (RCA) may correlate with future changes in non-calcified plaque burden. In examples in which longitudinal CT data is utilized (e.g., where both baseline data and follow-up data is received for a population or for a particular patient), statistical distributions of plaque progression in a large population of patients, in relation to measures of EAT or PCAT, may be used to aid in predicting plaque progression for a particular patient. Such statistical distributions may further be used to train a machine-learning model. For example, contrast CT may be used, and plaque progression may be predicted using information related to, for example, EAT volume or information related to PCAT attenuation. As another example, non-contrast CT may be used, lesion-based PCAT measurements may be obtained, and the PCAT measurements may aid in detecting non-calcified plaque.

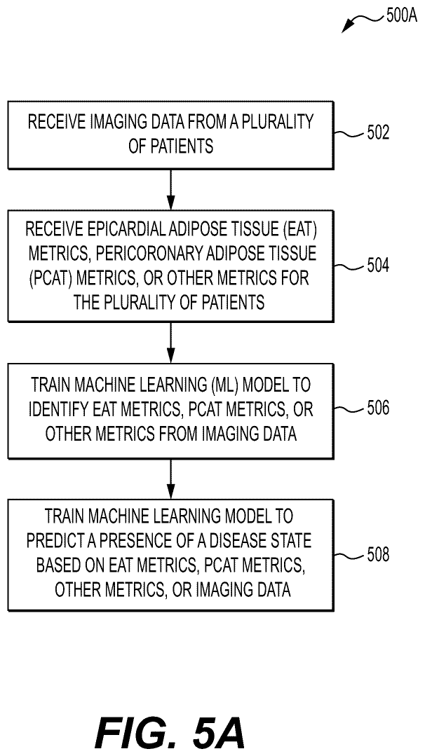

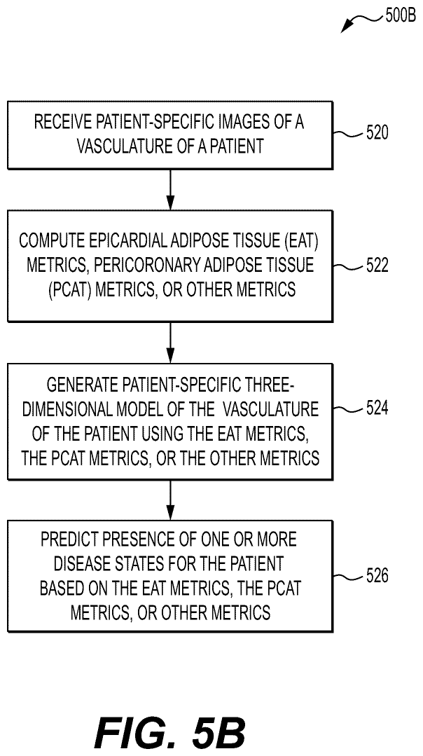

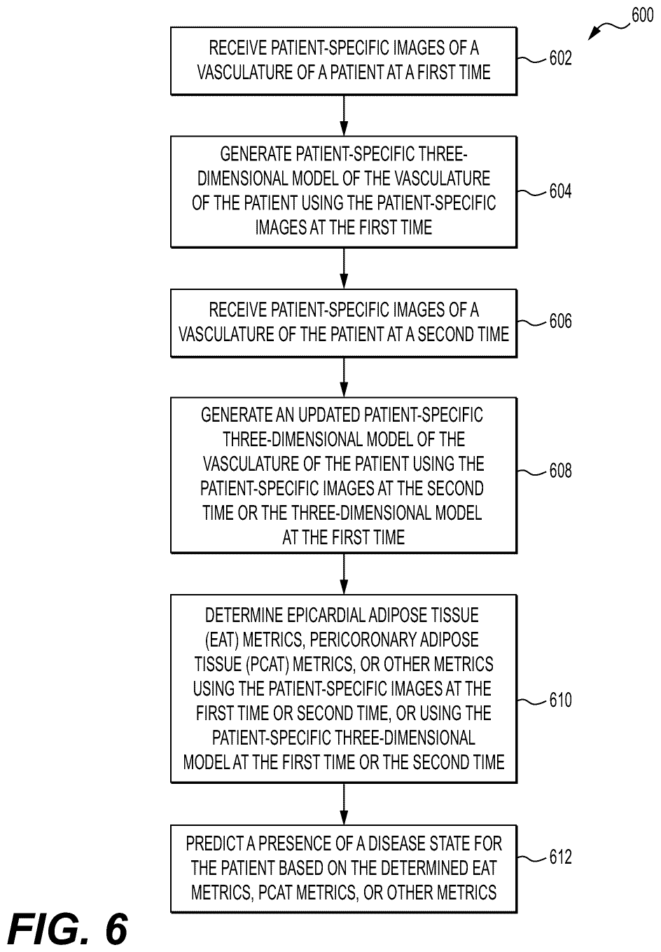

Still further, and as will be described in reference to FIGS. 5A-5B and 6, the systems and methods described herein may be utilized to predict a likelihood or a presence of a disease state for a patient or a subject. One example of such a disease state is ischemia with no obstructive arteries (INOCA). High PCAT attenuation may be linked to impaired ischemia with no obstructive CAD as measured by Doppler coronary flow velocity reserve (CFVR) and PET. For example, when contrast CT is used, information related to EAT (e.g., volume) or PCAT (e.g., attenuation) may be used to predict a presence of a disease state such as INOCA. As another example, when non-contrast CT is used, PCAT may be measured and used to predict a presence of a disease state, e.g., INOCA.

As mentioned, using the systems and methods described herein, measurements relating to EAT and PCAT may be utilized to provide added value over or in combination with existing imaging-based features and clinical risk factors. For example, EAT volume and mean PCAT attenuation in proximal main vessels may be used to stratify the risk of adverse cardiovascular events. Further, lesion-based PCAT attenuation has been shown to be higher for patients with ACS versus patients with stable coronary artery disease (CAD), and lesion-based PCAT attenuation has also been shown to be higher around soft plaque versus calcified plaque. Integration of image-based lesion-specific measures of plaque geometry, plaque type, or hemodynamics (or a combination thereof) with whole-heart EAT metrics and non-imaging-based risk factors may be used to determine which patients are at risk of a particular cardiovascular event or which lesions present more risk to a patient.

The disclosure provides systems and methods for utilizing EAT or PCAT information in evaluating patients. The disclosed systems and methods solve technological problems, including those arising from use of computers. For example, the disclosed systems and methods improve the technology of computerized modeling of coronary vessels. The medical images relating to EAT and PCAT described herein may be used in combination with clinical risk factors (e.g., PREVENT factors, atherosclerotic cardiovascular disease risk calculation, etc.,), plaque (e.g., plaque volumes, plaque burdens, calcium score, adverse plaque characteristics), and hemodynamics or computational physiology (FFRct (fractional flow reserve-computed tomography), delta FFRct, plaque stresses, etc.). These metrics may be referred to herein as “other metrics” or as “another metric.” Metrics obtained from the medical images relating to EAT and PCAT, and in some instances, the other metrics, may be used to provide a CT-centric risk score for a patient or subject or to classify a disease state.

As used herein, a “machine-learning model” generally encompasses instructions, data, or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine-learning techniques, such as linear regression, logistic regression, random forest, gradient boosted machine (GBM), deep learning, or a deep neural network. Supervised or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

While several of the examples herein reference certain types of machine-learning, it should be understood that techniques according to this disclosure may be adapted to any suitable type of machine-learning. It should also be understood that the examples above and below are illustrative only. The techniques and technologies of this disclosure may be adapted to any suitable activity.

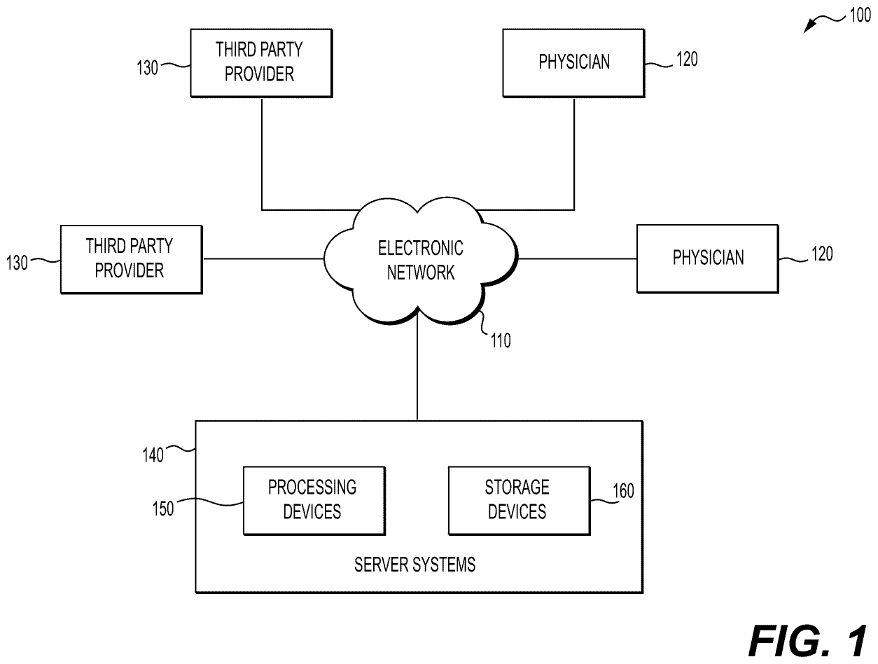

FIG. 1 depicts an example of an environment 100 in which such a computer system may be implemented as server systems 140. In addition to server systems 140, the environment of FIG. 1 further includes a plurality of physicians 120 and third party providers 130, any of which may be connected to an electronic network 110, such as the Internet, through one or more computers, servers, or handheld mobile devices. In FIG. 1, physicians 120 and third party providers 130 may each represent a computer system, as well as an organization that uses such a system. For example, a physician 120 may be a hospital or a computer system of a hospital.

Physicians 120 or third party providers 130 may create or otherwise obtain medical images, such as images of the cardiac, vascular, or organ systems, of one or more patients. Physicians 120 or third party providers 130 may also obtain any combination of patient-specific (or subject-specific) information, such as age, medical history, blood pressure, blood viscosity, and other types of patient-specific information. Physicians 120 or third party providers 130 may transmit the patient-specific information to server systems 140 over the electronic network 110.

Server systems 140 may include one or more storage devices 160 for storing images and data received from physicians 120 or third party providers 130. The storage devices 160 may be considered to be components of the memory of the server systems 140. Server systems 140 may also include one or more processing devices 150 for processing images and data stored in the storage devices and for performing any computer-implementable process described in this disclosure. Each of the processing devices 150 may be a processor or a device that include at least one processor.

In some embodiments, server systems 140 may comprise or utilize a cloud computing platform with scalable resources for computations or data storage, and may run an application for performing methods described in this disclosure on the cloud computing platform. In such embodiments, any outputs may be transmitted to another computer system, such as a personal computer, for display or storage.

Other examples of computer systems for performing methods of this disclosure include desktop computers, laptop computers, and mobile computing devices such as tablets and smartphones.

A computer system, such as server systems 140, may include one or more computing devices. If the one or more processors of the computer system is implemented as a plurality of processors, the plurality of processors may be included in a single computing device or distribute among a plurality of computing devices. If a computer system comprises a plurality of computing devices, the memory of the computer system may include the respective memory of each computing device of the plurality of computing devices.

FIGS. 2A and 2B, as discussed above, provide example workflows 200A and 200B. Workflow 200A relates to training a machine learning model for analyzing aspects of plaque using EAT or PCAT. Workflow 200B relates to analyzing aspects of plaque using EAT or PCAT for a particular patient.

FIG. 2A depicts an exemplary workflow 200A for training a machine-learning model to predict progression of plaque or other aspects of plaque. As shown at step 202, imaging data may be received from a plurality of patients (or from a single patient) at a first time. The imaging data may be received or otherwise obtained using any suitable modality described herein, such as contrast CT, non-contrast CT, or spectral CT. The imaging may be imaging of a coronary or other vasculature.

As shown at step 204, workflow 200A may further include receiving data identifying EAT, PCAT, plaque, or other aspects in the imaging data at a first time. For example, step 204 may include receiving annotations created manually or automatically that identify EAT, PCAT, plaque or aspects of EAT, PCAT, or plaque (e.g., particular characteristics of the EAT, PCAT, or plaque). In example, the data received in step 204 may also identify other aspects of a vasculature. In aspects, plaque may be identified using other, previously trained, machine-learning models.

As shown at step 206, workflow 200A may further include receiving imaging data from a plurality of patients (or for a single patient) at a second time. The imaging data received in step 206 may have any of the properties of the imaging data received in step 202. In other words, workflow 200A may include longitudinal image data to allow training based on changes in EAT, PCAT, or plaque over time (e.g., for a given patient in the training population). It shall be understood, however, that workflow 200A may involve the use of the imaging data only at the first time and may not utilize any follow-up images that are received at a second time. As shown at step 208, workflow 200A may further include receiving data identifying EAT, PCAT, or plaque in the received imaging data at the second time. Step 208 may involve any of the aspects of step 204. Workflow 200A may also include repeating steps 202-208 to receive data from further times (e.g., third times, fourth times, etc.)

As shown at step 210, workflow 200A may further include training a machine-learning (ML) model to predict progression of plaque or other metrics based on the imaging data at the first time, the data identifying EAT, PCAT, or plaque at the first time, the imaging data at the second time, or the data identifying EAT, PCAT, or plaque at the second time. Additional metrics may be used to train the machine-learning algorithm, such as hemodynamic metrics or the other various metrics described herein. In other aspects, step 210 may include training a machine-learning model (a same or different machine-learning model) to identify aspects such as EAT, PCAT, or plaque from patient imaging (e.g., as described in step 306, below).

In regard to the training at step 210 of FIG. 2A, aspects of EAT or PCAT may be correlated during training with measured aspects of plaque in the received images. In some aspects, statistical distributions of plaque progression in a large population of patients may be utilized. For example, statistical distributions of plaque progression in a large population of patients may be related to measures of EAT and PCAT, and these distributions may be utilized in the model or to train the model. For example, mean PCAT attenuation measured around the right coronary may correlate with future changes in non-calcified plaque burden.

EAT and PCAT may be correlated with aspects of plaque and may be predictive of a progression or non-progression of plaque. For example, EAT (e.g., whole-heart EAT volume) and PCAT (e.g., PCAT attenuation) may correlate to (and may facilitate predictions regarding) plaque progression, severity, or outcomes. In aspects, PCAT may assist with detecting non-calcified plaque (e.g., in non-contrast CT images of a patient). In examples, PCAT may be used as an indicator to identify nearby soft plaque or vulnerable plaque. Mean PCAT attenuation measured, for example, around the right coronary artery may be predictive of future changes in non-calcified plaque burden. Mean PCAT attenuation may also be referred to as a Fat Attenuation Index (FAI). In other aspects, adipocyte size may be associated with a pericoronary FAI and used to predict plaque progression. FAI may also be used to predict changes to calcification volume or RCA plaque burden. FAI may be associated with lesions (e.g., plaques) that lead to adverse outcomes (e.g., myocardial infarctions (MI) or acute coronary syndrome (ACS)). A higher value of mean PCAT attenuation may be associated with plaque rupture vs. plaque erosion and may indicate a higher degree of inflammation in plaque rupture. In general, PCAT attenuation may be higher across culprit lesions as compared to non-culprit and stable lesions. Changes in RCA PCAT attenuation may be associated with changes in non-calcified plaque burden. For example, lesion-specific mean PCAT attenuation may be higher in lesions with non-calcified plaque as compared to calcified plaque. PCAT mean attenuation may be associated with stenosis severity. In other aspects, PCAT volume may differ in culprit lesions.

FIG. 2B depicts an exemplary workflow 200B for predicting or analyzing plaque progression. As shown at step 220, workflow 200B may include receiving patient-specific images of a vasculature (e.g., a coronary vasculature) of a patient at a first time. The patient-specific images of the vasculature of the patient may be received or otherwise obtained using any suitable technique or equipment described herein, such as contrast or non-contrast CT (e.g., pre-contrast and contrast CT). In aspects, workflow 200B may implement the machine learning model trained in workflow 200A in a production environment. In other aspects, workflow 200B may utilize a different model.

As shown at step 222, workflow 200B may further include generating a patient-specific three-dimensional model of the vasculature of the patient using the patient-specific images received at the first time. In some examples, step 222 may be omitted. For example, any of the aspects described in U.S. Pat. No. 8,734,356, issued May 27, 2014, may be utilized to generate the patient-specific three-dimensional model.

As shown at step 224, workflow 200B may further include receiving patient-specific images of a vasculature of the patient at a second time. The images received in step 224 may have any of the aspects of the images received at step 220. It shall be understood, however, that workflow 200B may involve the use of imaging data received only at the first time and may not utilize images that are received at a second time.

As shown at step 226, workflow 200B may further involve generating an updated patient-specific three-dimensional model of the vasculature of the patient using the patient-specific images at the second time or using the three-dimensional model at the first time. Step 226 may include any of the aspects of step 222. For example, the updated patient-specific model may reflect changes that occurred between the first time and the second time. In aspects, step 226 may be omitted.

As shown at step 228, workflow 200B may further include determining EAT, PCAT, plaque, or other relevant aspects using the patient-specific images received at the first time or the second time, or using the patient-specific three-dimensional model at the first time or the second time. For example, step 228 may include using the machine learning model trained in workflow 200A. In alternatives, step 228 may include using an additional machine learning model that has been separately trained to identify plaque, EAT, PCAT, plaque, or other aspects.

Any of the following methods may be used to identify EAT or PCAT in any of the methods disclosed herein. In aspects, PCAT attenuation may be indicated by conventional CT attenuation, virtual mono-energetic images (e.g., at 40 keV), slope of a spectral attenuation curve, or effective atomic number from spectral detector CT. For example, adipose tissue may be identified based on Hounsfield Units (HU). For example, PCAT may have values from −190 HU to −30 HU in CT. In examples, RCA PCAT volume and attenuation may be measured from pre-contrast CT. PCAT may enhance by a number of HU with contrast (e.g., approximately 3-6 HU). PCAT may be defined as a region of EAT some diameter away from the coronary artery outer wall. For example, to avoid blooming artifacts from contrast, a 1 mm gap may be left. PCAT may be considered in proximal main vessels, such as the RCA. PCAT thickness, volume, mean attenuation (FAI), volumetric perivascular characterization index, PCAT lesion-based attenuation, PCAT attenuation gradient, or PCAT radiomic features may be measured.

EAT may have values from −190 HU to −30 HU in CT. EAT total volume, thickness (e.g., right ventricle (RV) free wall, atrioventricular (AV) or interventricular (IV) grooves), cross-sectional area (e.g., along a short axis plane or long axis plane), or mean attenuation may be measured.

As shown at step 230, workflow 200B may further include predicting or analyzing plaque progression based on the determined EAT, PCAT, or plaque. Step 230 may include applying the machine-learning model trained in workflow 200A.

Step 230 may also include outputting a graphical representation of the analysis. For example, step 230 may include outputting a color-coded display identifying aspects of plaque, EAT, or PCAT with color-coded values or shading. For example, higher-risk areas (e.g., plaques) may include different colors of shading than lower-risk areas (e.g., plaques). The visual display may also include a simulated display of plaque at a future time. The display may incorporate the analysis of the EAT and PCAT aspects. In aspects, step 230 may include using PCAT or EAT to predict progression/changes in plaque over time based only on a baseline image (i.e., based only on an image of a patient obtained at a first time, omitting steps 224 and 226). Step 230 may include displaying a two-dimensional or a three-dimensional representation of a patient-specific model. In some aspects, a user may manipulate the model by, for example, rotating the model or zooming in on the model. In some aspects, the display may have multiple panes that simultaneously update based on the user's interaction.

As discussed above, EAT and PCAT may be correlated with aspects of plaque and may be predictive of a progression or non-progression of plaque. For example, EAT (e.g., whole-heart EAT volume) and PCAT (e.g., PCAT attenuation) may correlate to (and may facilitate predictions regarding) plaque progression, severity, or outcomes. In aspects, PCAT may assist with detecting non-calcified plaque (e.g., in non-contrast CT images of a patient). In examples, PCAT may be used as an indicator to identify nearby soft plaque or vulnerable plaque. Mean PCAT attenuation measured, for example, around the right coronary artery may be predictive of future changes in non-calcified plaque burden. In other aspects, adipocyte size may be associated with a pericoronary fat attenuation index (FAI) and used to predict plaque progression. FAI may also be used to predict changes to calcification volume or RCA plaque burden. FAI may be associated with lesions (e.g., plaques) that lead to adverse outcomes (e.g., myocardial infarctions (MI) or acute coronary syndrome (ACS)). A higher value of mean PCAT attenuation may be associated with plaque rupture vs. plaque erosion and may indicate a higher degree of inflammation in plaque rupture. In general, PCAT attenuation may be higher across culprit lesions as compared to non-culprit and stable lesions. Changes in RCA PCAT attenuation may be associated with changes in non-calcified plaque burden. For example, lesion-specific mean PCAT attenuation may be higher in lesions with non-calcified plaque as compared to calcified plaque. PCAT mean attenuation may be associated with stenosis severity. In other aspects, PCAT volume may differ in culprit lesions.

Workflow 200B may be used to provide a patient with information regarding disease status in relation to other patients (e.g., in terms of individual metrics and overall outlook). In some aspects, workflow 200B may include providing an output that assesses plaque progression in relation to a nomogram of PCAT/EAT or other metrics. In aspects, workflow 200B may help to monitor beneficial changes due to medical therapy (e.g., when images of a patient are obtained at a first time and a second time).

FIG. 3A depicts an exemplary workflow 300A for training a machine-learning (ML) model to determine a risk score (for a patient or subject) based on EAT metrics, PCAT metrics, other metrics, or imaging data. As shown at step 302, workflow 300A may include receiving imaging data from a plurality of patients. Step 302 may involve any of the aspects of steps 202, 206, 220, or 224, discussed above. The imaging data may be received using any suitable technique or equipment described herein, such as contrast or non-contrast CT.

As shown at step 304, workflow 300A may further include receiving EAT metrics, PCAT metrics, or other metrics from the plurality of patients. For example, step 304 may include receiving annotations created manually or automatically that identify EAT, PCAT, plaque or aspects of EAT, PCAT, or plaque, or other metrics (e.g., risk factors, vessel geometry and morphology, hemodynamic metrics, such as FFR, plaque, calcium scores, clinical data, etc.). In examples, EAT and PCAT metrics may be computed globally or regionally (e.g., lesion-specific). In example, the data received in step 204 may also identify other aspects of a vasculature. In aspects, plaque, anatomical structures, or other risk factors may be identified using other, previously trained, machine-learning models.

Although not explicitly shown in FIG. 3A, steps 302 and 304 may be repeated to obtain images at later times (e.g., second times, third times, etc.). In such examples, workflow 300A may include longitudinal analysis over time.

As shown at step 306, workflow 300A may further include training a machine-learning model to identify EAT metrics, PCAT metrics, or other metrics from imaging data. Step 306 may have any of the aspects of step 210, or step 306 may be included in workflow 200A, as discussed above. For example, the above-discussed aspects of identifying EAT or PCAT may be utilized in step 306.

As shown at step 308, workflow 300A may further include training the machine-learning model to determine a risk score for the patient based on the EAT metrics, the PCAT metrics, the other metrics, or the imaging data.

As described above, EAT volume and mean PCAT attenuation in proximal main vessels may have independent value in stratifying risk of adverse cardiovascular events. Aspects of EAT and PCAT may be combined with existing imaging-based features (e.g., plaque, FFR, pressure, or other flow metrics) or clinical risk factors. Any of the aspects of plaque, discussed above with respect to workflows 200A and 200B, may be utilized in step 308. Lesion-based PCAT attenuation may be higher in acute coronary syndrome (ACS) than in coronary artery disease (CAD) and around soft plaque as opposed to calcified plaque. Integration of image-based, lesion-specific measurements of PCAT, plaque geometry and type, and hemodynamics, together with patient-level whole-heart EAT metrics and non-imaging-based risk factors may be used to determine which patients are at risk of events, and which lesions are more at risk. CT inflammation imaging (generating, e.g., EAT metrics and PCAT attenuation) may be combined with hemodynamics/computational physiology (e.g., FFRct, delta FFRct, MAR, plaque stresses), clinical risk factors (e.g., PREVENT or atherosclerotic cardiovascular disease (ASCVD) risk calculation), or CT plaque imaging (e.g., plaque volumes, burden, calcium score, adverse plaque characteristics (ACP) to generate an overall risk score. Step 308 may include training a machine-learning model to consider, for example, the factors above, to generate a risk score.

FIG. 3B depicts an exemplary workflow 300B for computing a risk score for a patient or a subject. Workflow 300B, and a workflow 400, described below, may improve (e.g., via using attenuation of PCAT) risk stratification above the current state of the art risk scoring, which uses age, sex, risk factors (hypertension, hypercholesterolemia, diabetes mellitus, smoker status, epicardial adipose tissue volume), modified Duke coronary artery disease index, or number of high-risk plaque features on coronary CTA.

As shown at step 320, workflow 300B may include receiving patient-specific images of a vasculature of a patient. The patient-specific images of the vasculature of the patient may be received using any suitable technique or equipment described herein. Any of the aspects of steps 202, 206, 220, or 224 may be utilized.

As shown at step 322, workflow 300B may further include computing EAT metrics, PCAT metrics, or other metrics, e.g., using the patient-specific images of the vasculature of the patient. For example, step 322 may include applying the machine-learning model trained in workflow 300A. For example, step 322 may include identifying and quantifying aspects of EAT, PCAT, or other metrics, such as, for example, calcium score, hemodynamic metrics (e.g., FFR), plaque characteristics (e.g., plaque composition and volume), clinical metrics (e.g., blood pressure, stress test results, etc.), information about treatment, etc. EAT and PCAT metrics may be determined globally or locally (e.g., on a lesion-by-lesion basis).

As shown at step 324, workflow 300B may further include generating a patient-specific three-dimensional model of the vasculature of the patient using the EAT metrics, the PCAT metrics, or the other metrics. However, in aspects, step 324 may be omitted. In some aspects, step 324 may be performed by the trained model of workflow 300A, by another trained model, or by other methods. Step 324 may involve any aspect of steps 222 or 226.

As shown at step 326, workflow 300B may further include integrating the EAT metrics, the PCAT metrics, or the other metrics to determine a risk score for the patient (or subject). For example, step 326 may include using the model trained in workflow 300A. The risk score may be determined using any of the aspects discussed above with respect to, for example, step 308. In some aspects, a risk score may be a score that represents another metric, such as a hemodynamic metric. For example, diameter stenosis, total plaque volume, and FAI may be combined to predict impaired myocardial perfusion (e.g., by analyzing FFR). In another example, plaque volumes and EAT volume may be combined to predict FFR.

For example, the use of contrast CT may facilitate risk stratification and prediction of adverse outcomes for patients and may provide added value over existing imaging-based features and clinical risk factors. As another example, the use of non-contrast CT may facilitate determinations regarding proximal main vessel PCAT and may provide added value over other factors, e.g., calcium scoring, hemodynamic features, etc. During the determination of the risk score at step 326, higher mean PCAT attenuation may be observed in acute coronary syndrome (ACS) lesions as opposed to SAP (serum amyloid P) lesions. Further, higher mean PCAT attenuation may be associated with ACS lesions along with thin-cap fibroatheroma (TCFA) and macrophage presence. Still further, during the computation of the risk score at step 326, mean PCAT attenuation may be correlated with ruptured (or likely-to-rupture) lesions versus eroded (or likely-to-erode) lesions to facilitate determinations of patient risk score in regard to ACS versus CAD.

The results of step 326 may be output in a visual display. For example, the output may include a three-dimensional or two-dimensional report. The report may, for example, depict the model. In some aspects, the model may be manipulated (e.g., rotated, zoomed in on, etc.), with coordination across multiple panes of a display, which may be simultaneously updated as the user manipulates the model.

FIG. 4 depicts an exemplary workflow 400 that may also be used to determine a risk score for a patient or a subject. As shown at step 402, workflow 400 may include receiving patient-specific images of a vasculature of a patient at a first time. Step 402 may include any of the aspects described for steps 202, 206, 220, 224, 302, or 320. The patient-specific images may be received using any suitable technique or equipment described herein.

As shown at step 404, workflow 400 may further include generating a patient-specific three-dimensional model of the vasculature of the patient using the patient-specific images at the first time. Step 404 may include any aspect of steps 222 or 226.

As shown at step 406, workflow 400 may further include receiving patient-specific images of the vasculature of the patient at a second time. Step 406 may include any of the aspects described for steps 202, 206, 220, 224, 302, 320, or 402. In aspects, the images received in step 406 may be received after a treatment or other intervention. The analysis of the images received in step 406 may aid in evaluating the effectiveness of the treatment or intervention.

As shown at step 408, workflow 400 may further include generating an updated-patient-specific three-dimensional model of the vasculature of the patient using the patient-specific images at the second time or using the three-dimensional model at the first time. Step 408 may also be based, at least in part, on the patient-specific model generated in step 404. Step 408 may include any aspect of steps 222, or 226, 324, or 404. The patient-specific model generated in step 408 may reflect changes in risk to a patient, due to passage of time or due to treatment interventions.

As shown at step 410, workflow 400 may further include determining EAT metrics, PCAT metrics, or other metrics using the patient-specific images at the first time or the second time, or using the patient-specific three-dimensional model at the first time or the second time. In some aspects, step 410 may also be performed between steps 404 and 406. Step 410 may include applying the model trained in workflow 300A, and may include any aspect of step 322.

As shown at 412, workflow 400 may further include predicting or analyzing a risk score for the patient (or subject) based on the determined EAT metrics, PCAT metrics, or the other metrics. Step 412 may include applying the machine-learning model trained in workflow 300A. Step 412 may include any aspect of step 326, described above.

FIG. 5A depicts an exemplary workflow 500A for training a machine-learning model to predict a presence of a disease state for a patient or a subject. As shown at step 502, workflow 500A may include receiving imaging data from a plurality of patients. Step 502 may include any aspect of steps 202, 206, 220, 224, 302, 320, 402, or 406. In some aspects, the images may be obtained from patients known to be without CAD but with impaired coronary flow reserve (CFR) as determined by, for example, computed tomographic perfusion (CTP) imaging, PET imaging, or by invasive measurements. Furthermore, non-invasive metrics may be obtained, and these non-invasive metrics may be used to identify and characterize INOCA patients. Examples of non-invasive metrics may include microvascular resistance reserve (MRR), IMR (index of microvascular resistance), CFR, CFVR, hMR (hyperemic microvascular resistance), and RRR (resistive reserve ratio).

As shown at 504, workflow 500A may further include receiving EAT metrics, PCAT metrics, or other metrics for the plurality of patients. The other metrics may include, for example, vessel geometry or morphology, plaque volume, plaque composition, or hemodynamic values (e.g., FFR). However, the metrics are not so limited and may include any of the metrics described above. For example, step 504 may include receiving annotations created manually or automatically that identify EAT, PCAT, plaque or aspects of EAT, PCAT, or plaque (e.g., particular characteristics of the EAT, PCAT, or plaque). In example, the data received in step 204 may also identify other aspects of a vasculature. In aspects, features and metrics may be identified using other, previously trained, machine-learning models. Step 504 may include any of the aspects of steps 204, 208, or 304, described above.

As shown at step 506, workflow 500A may further include training a machine-learning model to identify EAT metrics, PCAT metrics, or other metrics from the imaging data. Step 506 may include any of the aspects of step 306, described above. In aspects, step 506 may be omitted, and other, previously trained models or other methods may be used to identify the metrics discussed in workflow 500B, below.

As shown at 508, workflow 500A may further include training the machine-learning model to predict a presence of a disease state based on the EAT metrics, the PCAT metrics, the other metrics, the three-dimensional models or the imaging data. The disease state predicted by the machine-learning model may include ischemia with no obstructive arteries (INOCA), heart failure, impaired myocardial perfusion or impaired microvascular dilation in local vessel territory or globally, hypertrophic cardiomyopathy, MI or risk for MI, stable CAD, or another cardiovascular disease state. Regarding impaired myocardial perfusion or impaired microvascular dilation, an inflammation index may be used locally or regionally to leverage regions without large vessels and to perform regional analysis of PCAT/EAT to determine regional coronary microvascular dysfunction status and to determine regional inflammation in relation to other forms of disease.

For example, PCAT attenuation may be higher in patients with impaired ischemia with no obstructive CAD, as measured by Doppler CFVR and PET. Step 508 may include training the machine-learning model to focus on regions of patient images with a high likelihood of vessels but where vessels are not fully resolved in CT. A regional analysis of PCAT attenuation may be performed by the machine-learning model to determine regional coronary microvascular dysfunction (CMD). Microvascular disease may be identified using an inflammation index locally or regionally. A regional analysis of PCAT or EAT (e.g., per lesion, per territory, per chamber, or per downstream region of a target vessel) may be used to determine regional CMD status or regional inflammation in relation to other forms of disease. Higher attenuation of PCAT may be associated with greater risk of MI, as opposed to in patients with stable CAD or without CAD. Furthermore, higher attenuation of PCAT may be associated with ACS, including Non-ST-Segment Elevation Myocardial Infarction (NSTEMI) ACS. For example, PCAT attenuation (e.g., in conjunction with thin-cap fibroatheroma (TCFA) or macrophage presence) may be higher in ACS lesions than instable angina pectoris (SAP) lesions. PCAT attenuation and non-calcified plaque volume (NCPV) may be associated with CFR. In patients with low coronary calcium scoring and without obstructive CAD (<50% stenosis), PCAT attenuation may be higher when CFR is lower. PCAT attenuation may also be correlated with coronary flow velocity reserve of the left anterior descending coronary artery.

FIG. 5B depicts an exemplary workflow 500B for predicting a presence of one or more disease states for a patient or for a subject. As shown at step 520, workflow 500B may include receiving patient-specific images of a vasculature of a patient. Step 520 may include any aspect of steps 202, 206, 220, 224, 302, 320, 402, 406, or 502. In some aspects, step 520 may involve gathering data (e.g., patient-specific images) from a patient without obstructive coronary artery disease, but with impaired CFR as determined by another invasive or non-invasive technique (e.g., CTP or PET). In some aspects, patients suspected of having CMD may be identified by invasive MRR. In other aspects, step 520 may be performed on any patient, without the above aspects, and these filters on patients may be used only during workflow 500A.

As shown at step 522, workflow 500B may further include computing EAT metrics, PCAT metrics, or other metrics. For example, the machine-learning model trained in workflow 500A may be used to determine these metrics. The EAT and PCAT metrics may be determined globally or regionally. The other metrics may include, for example, vessel geometry or morphology, plaque volume or characteristics, or hemodynamic metrics (e.g., FFR, pressure, flow velocity, etc.). The above list is merely exemplary, and other metrics may also be calculated. Step 522 may include any aspect of steps 228, 230, 322, or 410.

As shown at step 524, workflow 500B may further include generating a patient-specific three-dimensional model of the vasculature of the patient using the EAT metrics, the PCAT metrics, or the other metrics. Step 524 may include any aspect of steps 222, 226, 324, 404, or 408.

As shown at step 526, workflow 500B may further include predicting or determining a presence (or absence) of one or more disease states for the patient (or subject) based on the EAT metrics, the PCAT metrics, or the other metrics. Step 526 may be performed by the trained model of workflow 500A. For example, step 526 may include identifying impaired myocardial perfusion or impaired microvascular dilation in a local vessel territory or globally. Step 526 may use an inflammation index to modify decreased microvascular response during hyperemia. Step 526 may also identify heart failure or hypertrophic cardiomyopathy. Step 526 may also predict INOCA. The above-listed conditions are merely exemplary, and step 526 may identify any of the exemplary conditions discussed herein, including those discussed above, with respect to step 508. Step 526 may make use of any of the associations discussed above, with respect to 508.

For example, when contrast CT is used, metrics pertaining to EAT and PCAT may correlate to (and may be used to predict) a presence of a disease state, such as ischemia with no obstructive arteries (INOCA). As another example, when non-contrast CT is used, metrics relating to EAT and PCAT may correlate to (and may be used to predict) a presence of a disease state, e.g., INOCA. The prediction of a disease state may focus on regions in the CT with a high likelihood of vessels but not resolved in CT. The prediction of a disease state may involve performing regional analysis of attenuation to determine regional coronary microvascular dysfunction (CMD).

Step 526 may also include creating a displayed image, such as a three-dimensional or a two-dimensional display. The displayed image may have any of the features of any of the images discussed above, with respect to workflows 200B, 300B, or 400. For example, the displayed image may include color-coding that identifies severity of disease or likelihood of disease regionally or globally.

FIG. 6 depicts an exemplary workflow 600 for predicting a presence of one or more disease states for a patient or subject. As shown at step 602, workflow 600 may include receiving patient-specific images of a vasculature of a patient at a first time. Step 602 may include any of the aspects described for steps 202, 206, 220, 224, 302, 320, 402, 406, 502, or 520.

As shown at step 604, workflow 600 may further include generating a patient-specific three-dimensional model of the vasculature of the patient using the patient-specific images at the first time. Step 604 may include any aspect of steps 222, 226, 324, 404, 408, or 524.

As shown at step 606, workflow 600 may further include receiving patient-specific images of the vasculature of the patient at a second time. The images received at the second time may facilitate longitudinal analysis for a specific patient. Step 606 may include any of the aspects described for steps 202, 206, 220, 224, 302, 320, 402, 406, 502, or 520. It shall be understood, however, that workflow 600 may involve the use of the imaging data only at the first time and may not utilize any follow-up images that are received at a second time. In aspects, the images received in step 606 may be received after a treatment or other intervention. The analysis of the images received in step 606 may aid in evaluating the effectiveness of the treatment or intervention.

As shown at step 608, workflow 600 may further include generating an updated patient-specific three-dimensional model of the vasculature of the patient using the patient-specific images at the second time or using the three-dimensional model at the first time. Step 608 may also be based, at least in part, on the patient-specific model generated in step 604. Step 608 may include any aspect of steps 222, 226, 324, 404, 408, 524, or 604.

As shown at step 610, workflow 600 may further include determining EAT metrics, PCAT metrics, or other metrics using the patient-specific images at the first time or the second time, or using the patient-specific three-dimensional at the first time or the second time. In some aspects, step 610 may also be performed between steps 604 and 606. Step 610 may include applying the model trained in workflow 500A, and may include any aspect of steps 228, 322, 410, or 522.

As shown at step 612, workflow 600 may further include predicting a presence of a disease state for the patient (or subject) based on the determined EAT metrics, PCAT metrics, or the other metrics. Step 612 may include applying the machine-learning model trained in workflow 500A. Step 612 may include any aspect of step 526, described above.

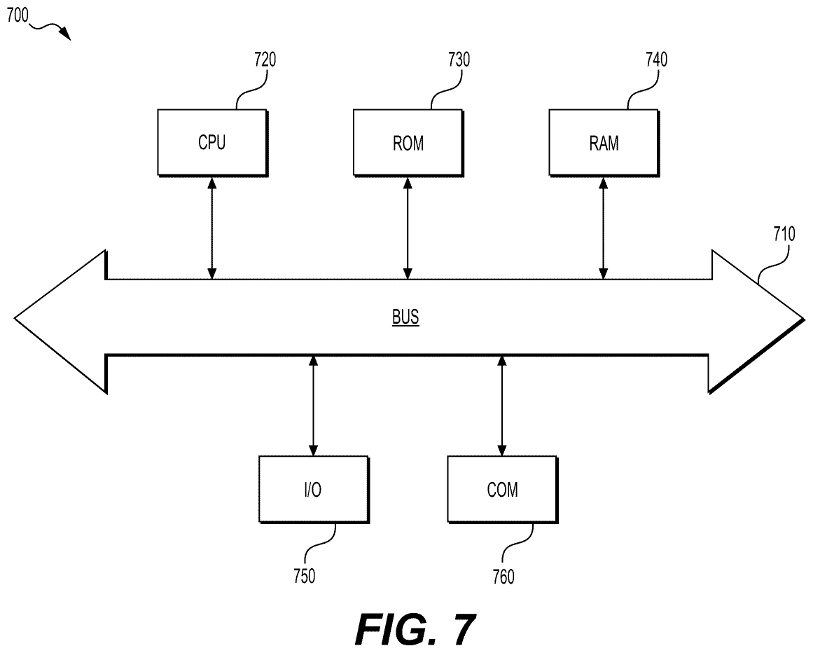

FIG. 7 depicts an exemplary system that may execute techniques presented herein. As shown in FIG. 7, device 700 may include a central processing unit (CPU) 720. CPU 720 may be any type of processor device including, for example, any type of special purpose or a general-purpose microprocessor device. As will be appreciated by persons skilled in the relevant art, CPU 720 also may be a single processor in a multi-core/multiprocessor system, such a system operating alone, or in a cluster of computing devices operating in a cluster or server farm. CPU 720 may be connected to a data communication infrastructure 710, for example a bus, message queue, network, or multi-core message-passing scheme.

Device 700 may also include a main memory 740, for example, random access memory (RAM), and also may include a secondary memory 730. Secondary memory 730, e.g. a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software or data.

In alternative implementations, secondary memory 730 may include similar means for allowing computer programs or other instructions to be loaded into device 700. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 700.

Device 700 also may include a communications interface (“COM”) 760. Communications interface 760 allows software and data to be transferred between device 700 and external devices. Communications interface 760 may include a modem, a network interface (such as an Ethernet card), a communications port, a PCMCIA slot and card, or the like. Software and data transferred via communications interface 760 may be in the form of signals, which may be electronic, electromagnetic, optical or other signals capable of being received by communications interface 760. These signals may be provided to communications interface 760 via a communications path of device 700, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 700 may also include input and output ports 750 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically may be grouped together to perform a function or group of related functions. Like reference numerals are generally intended to refer to the same or similar components. Components or modules may be implemented in software, hardware, or a combination of software or hardware.

The tools, modules, or functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

One or more techniques presented herein may enable a user, to better interact with a digital image of a glass slide that may be presented on a screen, in a virtual reality environment, in an augmented reality environment, or via some other form of visual display. One or more techniques presented herein may enable a natural interaction closer to traditional microscopy with less fatigue than using a mouse, keyboard, or other similar standard computer input devices.

The foregoing general description is exemplary and explanatory only, and not restrictive of the disclosure. Other embodiments of the invention may be apparent to those skilled in the art from consideration of the specification and practice of the invention disclosed herein. It is intended that the specification and examples be considered as exemplary only.

Instructions executable by one or more processors may also be stored on a non-transitory computer-readable medium. Therefore, whenever a computer-implemented method is described in this disclosure, this disclosure shall also be understood as describing a non-transitory computer-readable medium storing instructions that, when executed by one or more processors of a computer system, configure or cause the one or more processors to perform the computer-implemented method. Examples of non-transitory computer-readable media include random-access memory (RAM), read-only memory (ROM), solid-state storage media (e.g., solid state drives), optical storage media (e.g., optical discs), and magnetic storage media (e.g., hard disk drives). A non-transitory computer-readable medium may be part of the memory of a computer system or separate from any computer system.

A computer system may include one or more computing devices. If a computer system includes a plurality of processors, the plurality of processors may be included in a single computing device or distributed among a plurality of computing devices. A processor may be a central processing unit (CPU), a graphics processing unit (GPU), or another type of processing unit. The term “computational device,” as used in this disclosure, is interchangeable with “computing device.” An “electronic storage device” may include any of the non-transitory computer-readable media described above.

What is claimed is:

1. A computer-implemented method for processing medical images, the method comprising:
receiving image data for a patient;
based on the received image data, determining:
a patient-specific epicardial adipose tissue (EAT) metric or a patient-specific pericoronary adipose tissue (PCAT) metric; and
at least one other patient-specific metric; and
using the EAT metric or the PCAT metric, and the at least one other patient-specific metric, to determine a risk score for the patient or to classify a disease state of the patient.

2. The computer-implemented method of claim 1, wherein the at least one other patient-specific metric includes a vessel geometry, a vessel morphology, a plaque characteristic, or a hemodynamic measurement.

3. The computer-implemented method of claim 1, wherein the image data is a first image data, the EAT metric is a first EAT metric, the PCAT metric is a first PCAT metric, and the at least one other patient-specific metric is at least a first other patient-specific metric, and wherein, before determining the risk score or classifying the disease state of the patient, the computer-implemented method further comprises:
receiving second image data for the patient;
based on the received second image data, determining:
a second patient-specific EAT metric or a second patient-specific PCAT metric; and
at least a second other patient-specific metric; and
using the second EAT metric or the second PCAT metric, and the second other patient-specific metric to determine the risk score for the patient or to classify the disease state of the patient.

4. The computer-implemented method of claim 1, wherein the received image data is used to generate a three-dimensional model of a vasculature of the patient.

5. The computer-implemented method of claim 4, further comprising generating a display image of the three-dimensional model, wherein the display image includes a color-coded indicator of the risk score or the disease state.

6. The computer-implemented method of claim 1, wherein the disease state is Ischemia with Non-Obstructive Coronary Arteries (INOCA).

7. The computer-implemented method of claim 1, wherein the risk score is predictive of a fractional flow reserve (FFR) value.

8. The computer-implemented method of claim 1, wherein both the EAT metric and the PCAT metric are used to determine the risk score for the patient or to classify the disease state of the patient.

9. A system for processing medical images of a patient, comprising:
a data storage device storing instructions for medical image processing; and
a processor configured to execute the instructions to perform operations comprising:
receiving medical images of the patient;
based on the received medical images, determining:
a patient-specific epicardial adipose tissue (EAT) metric or a patient-specific pericoronary adipose tissue (PCAT) metric; and
at least one other patient-specific metric; and
using the EAT metric or the PCAT metric, and the at least one other patient-specific metric, to determine a risk score for the patient or to classify a disease state of the patient.

10. The system of claim 9, wherein the at least one other patient-specific metric includes a vessel geometry, a vessel morphology, a plaque characteristic, or a hemodynamic measurement.

11. The system of claim 9, wherein the received image data is used to generate a three-dimensional model of a vasculature of the patient.

12. The system of claim 11, wherein the system is further configured to generate a display image of the three-dimensional model, and wherein the display image includes a color-coded indicator of the risk score or the disease state.

13. The system of claim 9, wherein the disease state is Ischemia with Non-Obstructive Coronary Arteries (INOCA).

14. The system of claim 9, wherein the risk score is predictive of a fractional flow reserve (FFR) value.

15. The system of claim 9, wherein both the EAT metric and the PCAT metric are used to determine the risk score for the patient or to classify the disease state of the patient.

16. The system of claim 9, wherein the image data are a first image data, the EAT metric is a first EAT metric, the PCAT metric is a first PCAT metric, and the at least one other patient-specific metric is at least a first other patient-specific metric, and wherein, before determining the risk score or classifying the disease state of the patient, the processor is further configured to execute the instructions to perform operations comprising:
receiving second image data for the patient;
based on the received second image data, determining:
a second patient-specific EAT metric, or
a second patient-specific PCAT metric; and
at least a second other patient-specific metric; and
using the EAT metric or the second PCAT metric, and the second other patient-specific metric, to determine the risk score for the patient or to classify the disease state of the patient.

17. A non-transitory computer-readable medium storing instructions that, when executed by one or more processors, cause the one or more processors to perform a computer-implemented method for medical image processing, the method comprising:
receiving image data for a patient;
based on the received image data, determining:
a patient-specific epicardial adipose tissue (EAT) metric or a patient-specific pericoronary adipose tissue (PCAT) metric; and
at least one other patient-specific metric; and
using the EAT metric or the PCAT metric, and the at least one other patient-specific metric, to determine a risk score for the patient or to classify a disease state of the patient.

18. The non-transitory computer-readable medium of claim 17, wherein the at least one other patient-specific metric includes a vessel geometry, a vessel morphology, a plaque characteristic, or a hemodynamic measurement.

19. The non-transitory computer-readable medium of claim 17, wherein the received image data is used to generate a three-dimensional model of a vasculature of the patient.

20. The non-transitory computer-readable medium of claim 19, wherein the computer-implemented further involves generating a display image of the three-dimensional model, and wherein the display image includes a color-coded indicator of the risk score or the disease state.
